(19) United States
(12) Patent Application Publication
(10) Pub. No.: US 2023/0172928 A1
(43) Pub. Date: Jun. 8, 2023

(54) PHARMACEUTICAL COMBINATION COMPRISING TNO155 AND NAZARTINIB

(71) Applicant: Novartis AG, Basel (CH)

(72) Inventors: Huaixiang HAO, Lexington, MA (US); Chen LIU, Wayland, MA (US); Susan E. MOODY, Cambridge, MA (US)

(21) Appl. No.: 17/997,885

(22) PCT Filed: May 6, 2021
(86) PCT No.: PCT/IB2021/053867

§ 371 Date: Nov. 3, 2022

Related U.S. Application Data
(60) Provisional application No. 63/117,388, filed on Nov. 23, 2020.
(60) Provisional application No. 63/021,772, filed on May 8, 2020.

(51) Int. Cl. A61K 31/497; A61K 31/55; A61P 35/00

(52) U.S. Cl. CPC A61K 31/497; A61K 31/55; A61P 35/00

(57) ABSTRACT

The present invention relates to a pharmaceutical combination comprising TNO155 and nazartinib; pharmaceutical compositions comprising the same; and methods of using such combinations and compositions in the treatment or prevention of conditions in a SHP2 inhibitor combined with EGFR inhibition is beneficial in, for example, the treatment of cancers.

### FIELD OF THE INVENTION

The present invention relates to a pharmaceutical combination comprising TNO155 and nazartinib; pharmaceutical compositions comprising the same; and methods of using such combinations and compositions in the treatment or prevention of conditions in which SHP2 inhibition combined with EGFR inhibition is beneficial, for example, in the treatment of cancers. The present invention also relates to TNO155, or a pharmaceutically salt thereof, for use in treating cancer, wherein TNO155. or a pharmaceutically salt thereof, is co-administered with nazartinib, or a pharmaceutically acceptable salt thereof. The present invention also relates to nazartinib, or a pharmaceutically salt thereof, for use in treating cancer, wherein nazartinib or a pharmaceutically salt thereof, is co-administered with TNO155, or a pharmaceutically salt thereof.

### BACKGROUND OF THE INVENTION

Aberrant receptor tyrosine kinase (RTK) signaling is a common feature of many human cancers, frequently leading to sensitivity to therapies targeting these kinases. Examples of such cancers include EGFR-mutant lung cancers, KIT-mutant gastrointestinal stromal tumors (GISTs), and HER2-positive breast cancers, as well as head and neck squamous cell carcinomas (HNSCCs) and RAS/BRAF-WT colorectal cancers (CRCs), both of which frequently overexpress EGFR. SHP2 is a phosphatase that binds activated RTKs and transduces their signaling downstream to the Ras/MAPK and PI3K/Akt pathways. Inhibition of SHP2 therefore inhibits RTK-mediated signaling.

SHP2 has also been described to regulate PI3K, Fak, RhoA, Ca2+ oscillations, Ca2+/Calcineurin and NFAT signaling and SHP2 also acts downstream of cytokine signaling in the regulation of Jak/Stat signaling. In addition, SHP2 signals downstream of the immune checkpoint molecule PD-1, B- and T-lymphocyte attenuator (BTLA), and indoleamine 2,3-dioxygenase (IDO). Thus SHP2 can have RAS/MAPK-independent functions in tumorigenesis by regulating neoplastic migration, invasion, metastasis, or anti-tumor immune response.

Worldwide, lung cancer accounts for 13% (1.6 million) of all total cancer cases and 18% (1.4 million) of cancer deaths. In the US, lung cancer accounts for over 160,000 deaths per year. In Western countries, 10-15% of non-small cell lung cancer (NSCLC) patients harbor activating epidermal growth factor receptor (EGFR) mutations within their tumors (accounting for 20,000 to 30,000 new patients per year in the US), and Asian countries have reported rates as high as 30-40%. The predominant oncogenic EGFR mutations (L858R and ex19del) account for about 90% of EGFR-mutant NSCLC. This results in the activation of multiple pathways that promote survival, proliferation, angiogenesis and metastasis.

Patients with EGFR-mutant NSCLC have a high disease control rate with 1st generation EGFR inhibitors (e.g., erlotinib, gefitinib), but resistance invariably develops. Approximately 50% of resistant tumors harbor EGFR gatekeeper T790M mutations, while the other 50% harbor a variety of genetic alterations, which in many cases promote parallel signaling that converges on SHP2 (for example, amplification of MET, ERBB2, HGF). Furthermore, patients who develop EGFR T790M mutations have a high disease control rate with 3^{rd} generation EGFR inhibitors (for example, nazartinib and osimertinib), but resistance to these agents also develops. Resistance to these agents is less well-characterized, but in some cases has been found to be associated with EGFR C797S mutation, which disrupts binding of 3rd generation EGFR inhibitors, or amplification of MET or FGFR1. These findings highlight the continued addiction of these cancers to RTK signaling, which should predict sensitivity to SHP2 inhibition. No molecularly-targeted treatment options remain for these patients.

About 30% of NSCLCs harbor activating KRAS mutations, and these mutations are associated with resistance to EGFR TKIs. These mutations introduce an amino acid substitution at position 12, 13, or 61, and the G12C mutation is one of the most common KRAS mutations in lung cancer, found in about 12% of lung adenocarcinomas. Interestingly, EGFR and KRAS mutations are rarely detected in the same tumor, suggesting that they may perform functionally equivalent roles in lung tumorigenesis. Direct inhibition of KRAS has proven to be challenging with the exception of recent progress in targeting KRASG12C. Instead, inhibitors targeting the downstream signaling nodes of KRAS, such as RAF, MEK and ERK, have been developed and tested clinically in KRAS-mutant NSCLC as a single agent or in combinations. Despite these efforts, however, no targeted therapies are approved for patients with KRAS-mutant NSCLC.

Approximately 550,000 cases of head and neck carcinoma (HNSCC) are diagnosed annually worldwide. In the United States, approximately 50,000 cases are estimated to occur annually, with approximately one-third of patients dying within 5 years of diagnosis. Head and neck carcinomas are characterized by frequent amplification of EGFR, FGFRs, and their ligands, and approximately 90% are of squamous histology. In addition, the monoclonal antibody targeting EGFR, cetuximab, has demonstrated anti-tumor efficacy in metastatic/unresectable head and neck squamous cell carcinoma. However, disease control with cetuximab-containing regimens is relatively short-lived in this patient population, and few treatment options remain in this indication after progression on standard-of-care therapy. The high frequency of amplification or overexpression of RTK signaling components in HNSCC, in conjunction with preclinical data demonstrating a high rate of sensitivity of HNSCC cell lines to SHP2 suppression of inhibition, suggests that these cancers may be sensitive to SHP2 inhibition.

Approximately 232,000 new cases of skin melanoma are diagnosed globally each year, with an incidence that has been increasing steadily for several decades. The MAPK pathway plays a major role in the development and progression of melanoma. BRAF mutations occur in 40-60% and NRAS mutations occur in 15-20% of melanoma patients. These mutations constitutively activate BRAF and downstream signal transduction in the MAPK pathway, which signals for cancer cell proliferation and survival. The third most frequently mutated gene in the MAPK pathway in melanoma is NF1, which is mutated in ˜14% of melanoma, with more than half of its these mutations predicted to result in loss-of-function. NF1-mutated melanoma represents about half of BRAF and NRAS wild-type melanoma. NF1-mutated melanoma patients tend to have higher mutation burden and worse prognosis. Many of these patients do respond to PD-1 inhibitors, but unmet medical needs still exists for those who are refractory to or have relapsed on PD-1 inhibitor treatment.

Many other metastatic/unresectable solid malignancies display dependence on RTK signaling, such as KIT- or PDGFRA-mutant GISTs, which are frequently sensitive to imatinib, K/NRAS-WT CRCs, which may display sensitivity to cetuximab and panitumumab, medullary thyroid cancers, which are frequently sensitive to the RET-, VEGFR-, and EGFR-targeting TKI vandetanib or ALK-rearranged NSCLCs, which commonly respond to crizotinib or ceritinib. In cases in which mechanisms of resistance to such agents have been described, re-activation of RTK signaling is common and would be expected to predict sensitivity to SHP2 inhibition.

TNO155 is a selective, an orally bioavailable, allosteric inhibitor of wild-type SHP2. TNO155 has demonstrated significant efficacy in preclinical cancer models (in vitro and in vivo). In preclinical models, sensitivity to RTK suppression or inhibition predicted sensitivity to TNO155, whereas the presence of constitively activating mutations in RAS, BRAF or PTPN11 (gene encoding SHP2) predicted lack of sensitivity to TNO155. These observations are consistent with the role of SHP2 in RTK signaling upstream of RAS and BRAF, and the biochemical evidence that TNO155 inhibits wild-type SHP2. TNO155 demonstrated potent mitogen-activated protein kinase (MAPK) pathway pharmacodynamic modulation and anti-proliferative activity in cell lines and xenograft tumor models that are dependent on RTK signaling for survival and proliferation.

Nazartinib is a 3rd generation EGFR TKI that binds irreversibly to EGFR C797, and is active against EGFR sensitizing mutations (for example, ex19del, L858R) as well as the gatekeeper mutation T790M. Described resistance mechanisms to 1st through 3rd generation EGFR TKIs include mutations that render the mutant EGFR insensitive to the TKI, as well as activation of other RTK bypass pathways, such as MET or HGF amplification; resistance mechanisms may be heterogeneous even within a given tumor. Thus the combination of TNO155 with nazartinib can prevent or delay the emergence of many described resistance mechanisms, even in the context of heterogeneity.

The combination of the present invention, TNO155 and nazartinib, can overcome acquired resistance to EGFR inhibitors due to either secondary EGFR mutations or MET amplification. In addition, the combination of TNO155 and nazartinib is synergistic, coincident with sustained ERK inhibition and would be beneficial in the treatment of cancers selected from, but not limited to: EGFR-mutant non-small cell lung cancer (NSCLC); KRAS mutant non-small cell lung cancer; head and neck squamous cell carcinoma (HNSCC); melanoma; gastrointestinal stromal tumors (GIST); colorectal cancer (CRC); medullary thyroid cancers; and ALK-rearranged NSCLC.

### SUMMARY OF THE INVENTION

The present invention provides for a pharmaceutical combination comprising:

(a) (3S,4S)-8-(6-amino-5-((2-amino-3-chloropyridin-4-yl)thio)pyrazin-2-yl)-3-methyl-2-oxa-8-azaspiro[4.5]decan-4-amine (TNO155), or a pharmaceutically acceptable salt thereof, having the structure:

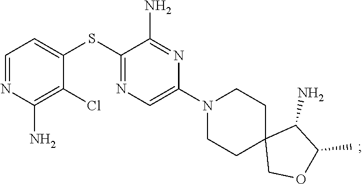 [SMILES: C[C@@H]1OCC2(CCN(c3cnc(Sc4ccnc(N)c4Cl)c(N)n3)CC2)[C@@H]1N]

and

(b) (R,E)-N-(7-chloro-1-(1-(4-(dimethylamino)but-2-enoyl)azepan-3-yl)-1H-benzo[d]imidazol-2-yl)-2-methylisonicotinamide (nazartinib), or a pharmaceutically acceptable salt thereof, having the structure:

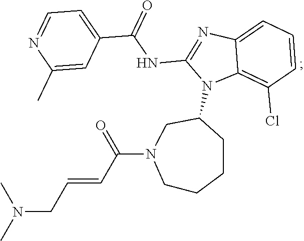 [SMILES: Cc1cc(C(=O)Nc2nc3cccc(Cl)c3n2[C@@H]2CCCCN(C(=O)/C=C/CN(C)C)C2)ccn1]

and

Combinations of TNO155, or a pharmaceutically acceptable salt thereof, and nazartinib, or a pharmaceutically acceptable salt thereof, will also be referred to herein as a “combination of the invention”.

In another embodiment of the combination of the invention, TNO155 or a pharmaceutically acceptable salt thereof and nazartinib, or a pharmaceutically acceptable salt thereof, are in the same formulation.

In another embodiment of the combination of the invention, TNO155 or a pharmaceutically acceptable salt thereof and nazartinib, or a pharmaceutically acceptable salt thereof, are in separate formulations.

In another embodiment, the combination of the invention is for simultaneous or sequential (in any order) administration.

In another embodiment is a method for treating or preventing cancer in a subject in need thereof comprising administering to the subject a therapeutically effective amount of the combination of the invention.

In a further embodiment of the method, the cancer is selected from: EGFR-mutant non-small cell lung cancer (NSCLC); KRAS mutant non-small cell lung cancer; head and neck squamous cell carcinoma (HNSCC); melanoma; gastrointestinal stromal tumors (GIST); colorectal cancer (CRC); medullary thyroid cancers; and ALK-rearranged NSCLC.

In a further embodiment of the method, the cancer is selected from EGFR-mutant non-small cell lung cancer (NSCLC).

In a further embodiment, the invention provides the combination of the invention for use in the treatment of a cancer, e.g. a cancer selected from: EGFR-mutant non-small cell lung cancer (NSCLC); KRAS mutant non-small cell lung cancer; head and neck squamous cell carcinoma (HNSCC); melanoma; gastrointestinal stromal tumors (GIST); colorectal cancer (CRC); medullary thyroid cancers; and ALK-rearranged NSCLC.

In a further embodiment, the invention provides for a combination of the invention for use in the manufacture of a medicament for treating a cancer selected from: EGFR-mutant non-small cell lung cancer (NSCLC); KRAS mutant non-small cell lung cancer; head and neck squamous cell carcinoma (HNSCC); melanoma; gastrointestinal stromal tumors (GIST); colorectal cancer (CRC); medullary thyroid cancers; and ALK-rearranged NSCLC.

In another embodiment is a pharmaceutical composition comprising the combination of the invention.

In a further embodiment, the pharmaceutical composition further comprises one or more pharmaceutically acceptable excipients.

### BRIEF DESCRIPTION OF THE DRAWINGS

FIG. 1: Combination dose matrix of TNO155 and nazartinib evaluating their anti-proliferative effects against PC-14 and NCI-H1975 cells. The mean (n=3) of percentages of inhibition (relative to the DMSO-treated control) of compound-treated cells in 6-day assays and the corresponding Loewe excess matrix are shown.

FIG. 2: Immunoblot of indicated proteins with lysates from PC-14 cells that were treated with nazartinib (0.1 or 0.3 μM), 3 μM TN0155, or the combination of nazartinib and TNO155 for 4 h or 24 h.

FIG. 3: Percentage change in tumor volumes of EGFR mutant NSCLC patient-derived xenografts in nude mice over time following treatment with vehicle, osimertinib (10 mg/kg of body weight (mpk), daily), TNO155 (10 mpk, twice daily), or the combination of osimertinib and TNO155.

### DEFINITIONS

The general terms used hereinbefore and hereinafter preferably have within the context of this disclosure the following meanings, unless otherwise indicated, where more general terms wherever used may, independently of each other, be replaced by more specific definitions or remain, thus defining more detailed embodiments of the invention:

The term “subject” or “patient” as used herein is intended to include animals, which are capable of suffering from or afflicted with a cancer or any disorder involving, directly or indirectly, a cancer. Examples of subjects include mammals, e.g., humans, apes, monkeys, dogs, cows, horses, pigs, sheep, goats, cats, mice, rabbits, rats, and transgenic non-human animals. In an embodiment, the subject is a human, e.g., a human suffering from, at risk of suffering from, or potentially capable of suffering from cancers.

The term “treating” or “treatment” as used herein comprises a treatment relieving, reducing or alleviating at least one symptom in a subject or effecting a delay of progression of a disease. For example, treatment can be the diminishment of one or several symptoms of a disorder or complete eradication of a disorder, such as cancer. Within the meaning of the present disclosure, the term “treat” also denotes to arrest, delay the onset (i.e., the period prior to clinical manifestation of a disease) and/or reduce the risk of developing or worsening a disease.

The terms “comprising” and “including” are used herein in their open-ended and non-limiting sense unless otherwise noted.

The terms “a” and “an” and “the” and similar references in the context of describing the invention (especially in the context of the following claims) are to be construed to cover both the singular and the plural, unless otherwise indicated herein or clearly contradicted by context. Where the plural form is used for compounds, salts, and the like, this is taken to mean also a single compound, salt, or the like.

As is customary in the art, dosages refer to the amount of the therapeutic agent in its free form. For example, when a dosage of 150 mg of nazartinib is referred to, and nazartinib is used as its mesylate salt, the amount of the therapeutic agent used is equivalent to 150 mg of the free form of nazartinib.

The terms“about” and “approximately” shall generally mean an acceptable degree of error for the quantity measured given the nature or precision of the measurements. Exemplary degrees of error are within 20 percent (%), typically, within 10%, and more typically, within 5% of a given value or range of values. In particular, where a dosage is mentioned as ‘about’ a particular value, it is intended to include a range around the specified value of plus or minus 10%.

In particular, where a dosage is mentioned as ‘about’ a particular value, or a dosage is referred to as a particular value (i.e. without the term “about” preceding that particular value), it is intended to include a range around the specified value of plus or minus 10%, or plus or minus 5%.

The term “combination therapy” or “in combination with” or “co-administered with” refers to the administration of two or more therapeutic agents to treat a condition or disorder described in the present disclosure (e.g., cancer). Such administration encompasses co-administration of these therapeutic agents in a substantially simultaneous manner, such as in a single capsule having a fixed ratio of active ingredients. Alternatively, such administration encompasses co-administration in multiple, or in separate containers (e.g., capsules, powders, and liquids) for each active ingredient. Powders and/or liquids may be reconstituted or diluted to a desired dose prior to administration. In addition, such administration also encompasses use of each type of therapeutic agent in a sequential manner, either at approximately the same time or at different times. In either case, the treatment regimen will provide beneficial effects of the drug combination in treating the conditions or disorders described herein.

The combination therapy can provide “synergy” and prove “synergistic”, i.e., the effect achieved when the active ingredients used together is greater than the sum of the effects that results from using the compounds separately. A synergistic effect can be attained when the active ingredients are: (1) co-formulated and administered or delivered simultaneously in a combined, unit dosage formulation; (2) delivered by alternation or in parallel as separate formulations; or (3) by some other regimen. When delivered in alternation therapy, a synergistic effect can be attained when the compounds are administered or delivered sequentially, e.g., by different injections in separate syringes. In general, during alternation therapy, an effective dosage of each active ingredient is administered sequentially, i.e., serially, whereas in combination therapy, effective dosages of two or more active ingredients are administered together.

The term “pharmaceutical combination” as used herein refers to either a fixed combination in one dosage unit form, or non-fixed combination or a kit of parts for the combined administration where two or more therapeutic agents may be administered independently at the same time or separately within time intervals, especially where these time intervals allow that the combination partners show a cooperative, e.g. synergistic effect.

The term “synergistic effect” as used herein refers to action of two therapeutic agents such as, for example, a compound TNO155 as a SHP2 inhibitor and nazartinib as an EGFR inhibitor, producing an effect, for example, slowing the symptomatic progression of a proliferative disease, particularly cancer, or symptoms thereof, which is greater than the simple addition of the effects of each drug administered by themselves. A synergistic effect can be calculated, for example, using suitable methods such as the Sigmoid-Emax equation (Holford, N. H. G. and Schemer, L. B., Clin. Pharmacokinet. 6: 429-453 (1981)), the equation of Loewe additivity (Loewe, S. and Muischnek, H., Arch. Exp. Pathol Pharmacol. 114: 313-326 (1926)) and the median-effect equation (Chou, T. C. and Talalay, P., Adv. Enzyme Regul. 22: 27-55 (1984)). Each equation referred to above can be applied to experimental data to generate a corresponding graph to aid in assessing the effects of the drug combination. The corresponding graphs associated with the equations referred to above are the concentration-effect curve, isobologram curve and combination index curve, respectively.

The combination of the invention, TNO155 and nazartinib, is also intended to represent unlabeled forms as well as isotopically labeled forms of the compounds. Isotopically labeled compounds have one or more atoms replaced by an atom having a selected atomic mass or mass number. Examples of isotopes that can be incorporated into TNO155 and nazartinib include isotopes of hydrogen, carbon, nitrogen, oxygen, and chlorine, for example, ²H, ³H, ¹¹C, ¹³C, ¹⁴C, ¹⁵N, ³⁵S ³⁶Cl. The invention includes isotopically labeled TNO155 and nazartinib, for example into which radioactive isotopes, such as ³H and ¹⁴C, or non-radioactive isotopes, such as ²H and ¹³C, are present. Isotopically labelled TNO155 and nazartinib are useful in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. Isotopically-labeled compounds of the invention can generally be prepared by conventional techniques known to those skilled in the art or by processes analogous to those described in the accompanying Examples using appropriate isotopically-labeled reagents.

Further, substitution with heavier isotopes, particularly deuterium (i.e., ²H or D) may afford certain therapeutic advantages resulting from greater metabolic stability, for example increased in vivo half-life or reduced dosage requirements or an improvement in therapeutic index. It is understood that deuterium in this context is regarded as a substituent of either TNO155 or nazartinib. The concentration of such a heavier isotope, specifically deuterium, may be defined by the isotopic enrichment factor. The term “isotopic enrichment factor” as used herein means the ratio between the isotopic abundance and the natural abundance of a specified isotope. If a substituent in TNO155 or nazartinib is denoted deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

### DESCRIPTION OF PREFERRED EMBODIMENTS

TNO155 is an investigational agent that is an orally bioavailable small molecule inhibitor of SHP2 activity. SHP2 transduces signaling downstream of activated RTKs. In preclinical models, tumor dependence on RTKs predicts dependence on SHP2.

In one embodiment is a method of treating cancer comprising administering to a subject in need thereof a pharmaceutical composition comprising (3S,4S)-8-(6-amino-5-((2-amino-3-chloropyridin-4-yl)thio)pyrazin-2-yl)-3-methyl-2-oxa-8-azaspiro[4.5]decan-4-amine, or pharmaceutically acceptable salt thereof, in combination with a second therapeutic agent.

In a further embodiment, the cancer is selected from: EGFR-mutant non-small cell lung cancer (NSCLC); KRAS mutant non-small cell lung cancer; head and neck squamous cell carcinoma (HNSCC); melanoma; gastrointestinal stromal tumors (GIST); colorectal cancer (CRC); medullary thyroid cancers; and ALK-rearranged NSCLC.

In a further embodiment, the cancer is in the advanced or metastatic stage.

In a further embodiment, the subject is a patient with either advanced NSCLC harboring an activating EGFR mutation and having progressed on standard-of-care (SOC) EGFR tyrosine kinase inhibitor (TKI) (or have no SOC EGFR TKI available) and having progressed on platinum-containing combination chemotherapy; or advanced NSCLC harboring a KRAS G12 mutation having progression on SOC; or advanced HNSCC having progressed on platinum-containing combination chemotherapy; or advanced esophageal SCC having progressed on platinum-containing chemotherapy; or advanced CRC lacking activating KRAS (with the exception of KRAS G12C), NRAS, or BRAF mutations and having progressed on fluoropyrimidine, oxaliplatin, and irinotecan; or advanced NRAS/BRAF WT cutaneous melanoma having progressed on SOC; or advanced GIST having progressed on SOC.

In a further embodiment, the subject is a patient suffering from one or more of the following cancers:

a. Advanced NSCLC harboring an EGFR TKI-sensitizing EGFR mutation (e.g., exon 19 deletion, L858R), after progression on osimertinib or nazartinib.

b. Advanced NSCLC harboring an EGFR TKI-sensitizing EGFR mutation (e.g., exon 19 deletion, L858R), after progression on a 1^{st} and/or 2nd generation EGFR TKI (e.g., erlotinib, gefitinib, afatinib) and which has been demonstrated to lack a T790 mutation following progression on these agents.

c. Advanced NSCLC harboring an EGFR TKI-sensitizing EGFR mutation (e.g., exon 19 deletion, L858R), progressing on osimertinib as the most recent prior therapy (continuing on osimertinib treatment until 2 weeks prior to starting study treatment (and thus osimertinib can be continued during the screening period) or such patients who recently discontinued osimertinib.

In a further embodiment, the cancer is EGFR-mutant non-small cell lung cancer (NSCLC).

In a further embodiment, the subject is a patient suffering from one or more of the following cancers:

a. Advanced NSCLC harboring an EGFR TKI-sensitizing EGFR mutation (e.g., exon 19 deletion, L858R), after progression on osimertinib or nazartinib.

b. Advanced NSCLC harboring an EGFR TKI-sensitizing EGFR mutation (e.g., exon 19 deletion, L858R), after progression on a 1^{st} and/or 2nd generation EGFR TKI (e.g., erlotinib, gefitinib, afatinib) and which has been demonstrated to lack a T790 mutation following progression on these agents.

c. Advanced NSCLC harboring an EGFR TKI-sensitizing EGFR mutation (e.g., exon 19 deletion, L858R), progressing on osimertinib as the most recent prior therapy (continuing on osimertinib treatment until 2 weeks prior to starting study treatment (and thus osimertinib can be continued during the screening period) or such patients who recently discontinued osimertinib.

In a further embodiment, the cancer is head and neck squamous cell carcinoma.

In a further embodiment, the cancer is KRAS mutant non-small cell lung cancer.

In a further embodiment, the cancer is head and neck squamous cell carcinoma (HNSCC).

In a further embodiment, the cancer is melanoma.

In a further embodiment, the cancer is gastrointestinal stromal tumors (GIST).

In a further embodiment, the cancer is colorectal cancer (CRC).

In a further embodiment, the cancer is medullary thyroid cancers.

In a further embodiment, the cancer is ALK-rearranged NSCLC.

In a further embodiment, (3S,4S)-8-(6-amino-5-((2-amino-3-chloropyridin-4-yl)thio)pyrazin-2-yl)-3-methyl-2-oxa-8-azaspiro[4.5]decan-4-amine, or pharmaceutically acceptable salt thereof, and the second therapeutic agent are administered simultaneously, separately or over a period of time.

In a further embodiment, the amount of (3S,4S)-8-(6-amino-5-((2-amino-3-chloropyridin-4-yl)thio)pyrazin-2-yl)-3-methyl-2-oxa-8-azaspiro[4.5]decan-4-amine, or pharmaceutically acceptable salt thereof, and the second therapeutic agent, administered to the subject in need thereof, is effective to treat the cancer.

In a further embodiment, the second therapeutic agent is an EGFR inhibitor.

In a further embodiment, the second therapeutic agent is osimertinib, or a pharmaceutically acceptable salt thereof.

In a further embodiment, osimertinib is administered orally at a dose ranging from about 40 mg to about 80 mg per day, with or without food.

In a further embodiment, osimertinib is administered orally as a dose of 80 mg per day, with or without food.

In a further embodiment, the EGFR inhibitor is (R,E)-N-(7-chloro-1-(1-(4-(dimethylamino)but-2-enoyl)azepan-3-yl)-1H-benzo[d]imidazol-2-yl)-2-methylisonicotinamide, or a pharmaceutically acceptable salt thereof.

In a further embodiment, (3S,4S)-8-(6-amino-5-((2-amino-3-chloropyridin-4-yl)thio)pyrazin-2-yl)-3-methyl-2-oxa-8-azaspiro[4.5]decan-4-amine is administered orally at a dose ranging from about 1.5 mg per day to about 100 mg per day, for example, from about 1.5 mg per day to about 60 mg per day or from about 20 mg per day to about 60 mg per day.

In a further embodiment, (3S,4S)-8-(6-amino-5-((2-amino-3-chloropyridin-4-yl)thio)pyrazin-2-yl)-3-methyl-2-oxa-8-azaspiro[4.5]decan-4-amine is administered orally at a dose of about 1.5 mg per day, or 3 mg per day, or 6 mg per day, or 10 mg per day, or 20 mg per day, or 30 mg per day, or 40 mg per day, or 50 mg per day, or 60 mg per day, or 70 mg per day, or 80 mg per day, or 90 mg per day, or 100 mg per day.

In a further embodiment, the dose (3S,4S)-8-(6-amino-5-((2-amino-3-chloropyridin-4-yl)thio)pyrazin-2-yl)-3-methyl-2-oxa-8-azaspiro[4.5]decan-4-amine, is administered on a cycle of 2 weeks off followed by 1 week off. Hence, in a further embodiment, (3S,4S)-8-(6-amino-5-((2-amino-3-chloropyridin-4-yl)thio)pyrazin-2-yl)-3-methyl-2-oxa-8-azaspiro[4.5]decan-4-amine is administered orally at a dose per day of 20 mg, 30 mg, 40 mg, 60 mg, 80 mg or 100 mg on a 21 day cycle of 2 weeks on drug followed by 1 week off drug.

In a further embodiment, (R,E)-N-(7-chloro-1-(1-(4-(dimethylamino)but-2-enoyl)azepan-3-yl)-1H-benzo[d]imidazol-2-yl)-2-methylisonicotinamide is administered orally at a dose ranging from about 75 mg per day to 350 mg per day.

In a further embodiment, (R,E)-N-(7-chloro-1-(1-(4-(dimethylamino)but-2-enoyl)azepan-3-yl)-1H-benzo[d]imidazol-2-yl)-2-methylisonicotinamide is administered orally at a dose of about 75 mg per day, or 100 mg per day, or 150 mg per day, or 200 mg per day, or 250 mg per day, or 300 mg per day, or 350 mg per day.

In a further embodiment, (R,E)-N-(7-chloro-1-(1-(4-(dimethylamino)but-2-enoyl)azepan-3-yl)-1H-benzo[d]imidazol-2-yl)-2-methylisonicotinamide is administered orally at 150 mg per day.

In another embodiment is a method of treating cancer comprising administering, to a patient in need thereof, (3S,4S)-8-(6-amino-5-((2-amino-3-chloropyridin-4-yl)thio)pyrazin-2-yl)-3-methyl-2-oxa-8-azaspiro[4.5]decan-4-amine is administered orally at a dose of about 1.5 mg per day, or 3 mg per day, or 6 mg per day, or 10 mg per day, or 20 mg per day, or 30 mg per day, or 40 mg per day, or 50 mg per day, or 60 mg per day, or 70 mg per day, or 80 mg per day, or 90 mg per day, or 100 mg per day.

In a further embodiment, (3S,4S)-8-(6-amino-5-((2-amino-3-chloropyridin-4-yl)thio)pyrazin-2-yl)-3-methyl-2-oxa-8-azaspiro[4.5]decan-4-amine is administered orally at a dose per day of 20 mg, 30, 40 or 60 mg on a 21 day cycle of 2 weeks on drug followed by 1 week off drug.

In a further embodiment, the cancer is selected from: EGFR-mutant non-small cell lung cancer (NSCLC); KRAS mutant non-small cell lung cancer; head and neck squamous cell carcinoma (HNSCC); melanoma; gastrointestinal stromal tumors (GIST); colorectal cancer (CRC); medullary thyroid cancers; and ALK-rearranged NSCLC.

In a further embodiment, the cancer is EGFR-mutant non-small cell lung cancer (NSCLC).

In a further embodiment, the cancer is head and neck squamous cell carcinoma.

In a further embodiment, the cancer is KRAS mutant non-small cell lung cancer.

In a further embodiment, the cancer is head and neck squamous cell carcinoma (HNSCC).

In a further embodiment, the cancer is melanoma.

In a further embodiment, the cancer is gastrointestinal stromal tumors (GIST).

In a further embodiment, the cancer is colorectal cancer (CRC).

In a further embodiment, the cancer is medullary thyroid cancers.

In a further embodiment, the cancer is ALK-rearranged NSCLC.

In a further embodiment, (3S,4S)-8-(6-amino-5-((2-amino-3-chloropyridin-4-yl)thio)pyrazin-2-yl)-3-methyl-2-oxa-8-azaspiro[4.5]decan-4-amine, or pharmaceutically acceptable salt thereof, and the second therapeutic agent are administered simultaneously, separately or over a period of time.

In a further embodiment, the second therapeutic agent is an EGFR inhibitor.

In a further embodiment, the second therapeutic agent is osimertinib, or a pharmaceutically acceptable salt thereof.

In a further embodiment, the EGFR inhibitor is (R,E)-N-(7-chloro-1-(1-(4-(dimethylamino)but-2-enoyl)azepan-3-yl)-1H-benzo[d]imidazol-2-yl)-2-methylisonicotinamide, or a pharmaceutically acceptable salt thereof.

In a further embodiment, (R,E)-N-(7-chloro-1-(1-(4-(dimethylamino)but-2-enoyl)azepan-3-yl)-1H-benzo[d]imidazol-2-yl)-2-methylisonicotinamide is administered orally at a dose of about 75 mg per day, or 100 mg per day, or 150 mg per day, or 200 mg per day, or 250 mg per day, or 300 mg per day, or 350 mg per day.

In a further embodiment, (R,E)-N-(7-chloro-1-(1-(4-(dimethylamino)but-2-enoyl)azepan-3-yl)-1H-benzo[d]imidazol-2-yl)-2-methylisonicotinamide is administered orally at 150 mg per day.

In one embodiment, with respect to the pharmaceutical combination of the invention, is a pharmaceutical combination comprising (3S,4S)-8-(6-amino-5-((2-amino-3-chloropyridin-4-yl)thio)pyrazin-2-yl)-3-methyl-2-oxa-8-azaspiro[4.5]decan-4-amine, or pharmaceutically acceptable salt thereof, and 7(R,E)-N-(7-chloro-1-(1-(4-(dimethylamino)but-2-enoyl)azepan-3-yl)-1H-benzo[d]imidazol-2-yl)-2-methylisonicotinamide, or a pharmaceutically acceptable salt thereof.

In a further embodiment, (3S,4S)-8-(6-amino-5-((2-amino-3-chloropyridin-4-yl)thio)pyrazin-2-yl)-3-methyl-2-oxa-8-azaspiro[4.5]decan-4-amine, or a pharmaceutically acceptable salt thereof, and (R,E)-N-(7-chloro-1-(1-(4-(dimethylamino)but-2-enoyl)azepan-3-yl)-1H-benzo[d]imidazol-2-yl)-2-methylisonicotinamide, or a pharmaceutically acceptable salt thereof, are administered separately, simultaneously or sequentially, in any order.

In a further embodiment, the pharmaceutical combination is for oral administration.

In a further embodiment, (3S,4S)-8-(6-amino-5-((2-amino-3-chloropyridin-4-yl)thio)pyrazin-2-yl)-3-methyl-2-oxa-8-azaspiro[4.5]decan-4-amine is in an oral dose form (hard gelatin capsules in dosage strength 1.5 mg, 5 mg, 10 mg and 50 mg).

In a further embodiment, (R,E)-N-(7-chloro-1-(1-(4-(dimethylamino)but-2-enoyl)azepan-3-yl)-1H-benzo[d]imidazol-2-yl)-2-methylisonicotinamide is in an oral dose form (hard gelatin capsules in a dosage strength of 25 mg or 50 mg).

In another embodiment, is a pharmaceutical composition comprising a pharmaceutical combination of (3S,4S)-8-(6-amino-5-((2-amino-3-chloropyridin-4-yl)thio)pyrazin-2-yl)-3-methyl-2-oxa-8-azaspiro[4.5]decan-4-amine, or pharmaceutically acceptable salt thereof, and (R,E)-N-(7-chloro-1-(1-(4-(dimethylamino)but-2-enoyl)azepan-3-yl)-1H-benzo[d]imidazol-2-yl)-2-methylisonicotinamide, or a pharmaceutically acceptable salt thereof and at least one pharmaceutically acceptable carrier.

In a further embodiment, is a pharmaceutical combination of (3S,4S)-8-(6-amino-5-((2-amino-3-chloropyridin-4-yl)thio)pyrazin-2-yl)-3-methyl-2-oxa-8-azaspiro[4.5]decan-4-amine, or pharmaceutically acceptable salt thereof, and (R,E)-N-(7-chloro-1-(1-(4-(dimethylamino)but-2-enoyl)azepan-3-yl)-1H-benzo[d]imidazol-2-yl)-2-methylisonicotinamide, or a pharmaceutically acceptable salt thereof, for use in the treatment of: EGFR-mutant non-small cell lung cancer (NSCLC); KRAS mutant non-small cell lung cancer; head and neck squamous cell carcinoma (HNSCC); melanoma; gastrointestinal stromal tumors (GIST); colorectal cancer (CRC); medullary thyroid cancers; and ALK-rearranged NSCLC.

In another embodiment, is a pharmaceutical combination of (3S,4S)-8-(6-amino-5-((2-amino-3-chloropyridin-4-yl)thio)pyrazin-2-yl)-3-methyl-2-oxa-8-azaspiro[4.5]decan-4-amine, or pharmaceutically acceptable salt thereof, and (R,E)-N-(7-chloro-1-(1-(4-(dimethylamino)but-2-enoyl)azepan-3-yl)-1H-benzo[d]imidazol-2-yl)-2-methylisonicotinamide, or a pharmaceutically acceptable salt thereof, for use in the treatment of EGFR-mutant non-small cell lung cancer (NSCLC).

In another embodiment, is a use of the pharmaceutical combination of ((3S,4S)-8-(6-amino-5-((2-amino-3-chloropyridin-4-yl)thio)pyrazin-2-yl)-3-methyl-2-oxa-8-azaspiro[4.5]decan-4-amine, or pharmaceutically acceptable salt thereof, and (R,E)-N-(7-chloro-1-(1-(4-(dimethylamino)but-2-enoyl)azepan-3-yl)-1H-benzo[d]imidazol-2-yl)-2-methylisonicotinamide, or a pharmaceutically acceptable salt thereof, for the manufacture of a medicament for the treatment of a cancer selected from: EGFR-mutant non-small cell lung cancer (NSCLC); KRAS mutant non-small cell lung cancer; head and neck squamous cell carcinoma (HNSCC); melanoma; gastrointestinal stromal tumors (GIST); colorectal cancer (CRC); medullary thyroid cancers; and ALK-rearranged NSCLC.

In another embodiment, is a method of treating a cancer selected from: EGFR-mutant non-small cell lung cancer (NSCLC); KRAS mutant non-small cell lung cancer; head and neck squamous cell carcinoma (HNSCC); melanoma; gastrointestinal stromal tumors (GIST); colorectal cancer (CRC); medullary thyroid cancers; and ALK-rearranged NSCLC; comprising administrating to a patient in need thereof a pharmaceutical combination of (3S,4S)-8-(6-amino-5-((2-amino-3-chloropyridin-4-yl)thio)pyrazin-2-yl)-3-methyl-2-oxa-8-azaspiro[4.5]decan-4-amine, or pharmaceutically acceptable salt thereof, and (R,E)-N-(7-chloro-1-(1-(4-(dimethylamino)but-2-enoyl)azepan-3-yl)-1H-benzo[d]imidazol-2-yl)-2-methylisonicotinamide, or a pharmaceutically acceptable salt thereof, or a pharmaceutical composition comprising a pharmaceutical combination of (3S,4S)-8-(6-amino-5-((2-amino-3-chloropyridin-4-yl)thio)pyrazin-2-yl)-3-methyl-2-oxa-8-azaspiro[4.5]decan-4-amine, or pharmaceutically acceptable salt thereof, and (R,E)-N-(7-chloro-1-(1-(4-(dimethylamino)but-2-enoyl)azepan-3-yl)-1H-benzo[d]imidazol-2-yl)-2-methylisonicotinamide, or a pharmaceutically acceptable salt thereof and at least one pharmaceutically acceptable carrier.

In another embodiment, is a method of treating a comprising cancer selected from: EGFR-mutant non-small cell lung cancer (NSCLC); KRAS mutant non-small cell lung cancer; head and neck squamous cell carcinoma (HNSCC); melanoma; gastrointestinal stromal tumors (GIST); colorectal cancer (CRC); medullary thyroid cancers; and ALK-rearranged NSCLC; administrating to a patient in need thereof a pharmaceutical combination of (3S,4S)-8-(6-amino-5-((2-amino-3-chloropyridin-4-yl)thio)pyrazin-2-yl)-3-methyl-2-oxa-8-azaspiro[4.5]decan-4-amine, or pharmaceutically acceptable salt thereof, and (R,E)-N-(7-chloro-1-(1-(4-(dimethylamino)but-2-enoyl)azepan-3-yl)-1H-benzo[d]imidazol-2-yl)-2-methylisonicotinamide, or a pharmaceutically acceptable salt thereof, or a pharmaceutical composition comprising a pharmaceutical combination of (3S,4S)-8-(6-amino-5-((2-amino-3-chloropyridin-4-yl)thio)pyrazin-2-yl)-3-methyl-2-oxa-8-azaspiro[4.5]decan-4-amine, or pharmaceutically acceptable salt thereof, and (R,E)-N-(7-chloro-1-(1-(4-(dimethylamino)but-2-enoyl)azepan-3-yl)-1H-benzo[d]imidazol-2-yl)-2-methylisonicotinamide, or a pharmaceutically acceptable salt thereof and at least one pharmaceutically acceptable carrier.

In another embodiment, (3S,4S)-8-(6-amino-5-((2-amino-3-chloropyridin-4-yl)thio)pyrazin-2-yl)-3-methyl-2-oxa-8-azaspiro[4.5]decan-4-amine is administered orally at a dose of about 1.5 mg per day, or 3 mg per day, or 6 mg per day, or 10 mg per day, or 20 mg per day, or 30 mg per day, or 40 mg per day, or 50 mg per day, or 60 mg per day.

In a further embodiment, (R,E)-N-(7-chloro-1-(1-(4-(dimethylamino)but-2-enoyl)azepan-3-yl)-1H-benzo[d]imidazol-2-yl)-2-methylisonicotinamide is administered orally at a dose of about 75 mg per day, or 100 mg per day, or 150 mg per day, or 200 mg per day, or 250 mg per day, or 300 mg per day, or 350 mg per day.

In a further embodiment, (R,E)-N-(7-chloro-1-(1-(4-(dimethylamino)but-2-enoyl)azepan-3-yl)-1H-benzo[d]imidazol-2-yl)-2-methylisonicotinamide is administered orally at a dose of about 150 mg per day, continuously.

### Pharmacology and Utility

Non-small cell lung cancer—In 2012, approximately 1.8 million people worldwide were diagnosed with lung cancer, and an estimated 1.6 million people died from the disease. Non-small cell lung cancer comprises approximately 85% of lung cancers, with adenocarcinomas and squamous cell carcinomas being the most common subtypes. Standard of care treatment for advanced stage non-small cell lung carcinomas (NSCLCs) that do not harbor genetic alterations in druggable driver oncogenes such as EGFR, ALK, or ROS includes chemotherapy and immunotherapy, administered concurrently or sequentially. While these treatments provide clinical benefit, the majority of patients experience disease progression within a year, and the prognosis for patients with advanced NSCLC remains poor. Immunotherapy for NSCLC with immune checkpoint inhibitors has demonstrated promise, with some NSCLC patients experiencing durable disease control for years. However, such long-term non-progressors are uncommon, and combination treatment strategies that can increase the proportion of patients responding to and achieving lasting remission with immunotherapy using checkpoint inhibitors are urgently needed. Activating mutations in the KRAS oncogene occur in approximately 30% of lung adenocarcinomas, and have been associated with poor outcome in some studies. No approved drugs target mutant KRAS directly, so standard of care for advanced stage KRAS-mutant NSCLC is also chemotherapy and immunotherapy as described above.

Approximately 232,000 new cases of skin melanoma are diagnosed globally each year, with an incidence that has been increasing steadily for several decades. The MAPK pathway plays a major role in the development and progression of melanoma. BRAF mutations occur in 40-60% and NRAS mutations occur in 15-20% of melanoma patients. These mutations constitutively activate BRAF and downstream signal transduction in the MAPK pathway, which signals for cancer cell proliferation and survival.

The third most frequently mutated gene in the MAPK pathway in melanoma is NF1, which is mutated in ˜14% of melanoma, with more than half of these mutations predicted to result in loss-of-function. NF1-mutated melanoma represents about half of BRAF and NRAS wild-type melanoma. NF1-mutated melanoma patients tend to have higher mutation burden and worse prognosis. Many of these patients do respond to PD-1 inhibitors, but unmet medical needs still exists for those who are refractory to or have relapsed on PD-1 inhibitor treatment.

TNO155 is a first-in-class allosteric inhibitor of wild-type SHP2. SHP2 is a ubiquitously expressed non-receptor protein tyrosine phosphatase (PTP) composed of two N-terminal SH2 domains, a classic PTP domain, and a C-terminal tail. The phosphatase activity is auto-inhibited by the two SHP2 domains that bind to the PTP domain (closed conformation). Upon activation of receptor tyrosine kinases (RTKs), SHP2 is recruited to the plasma membrane where it associates with activated RTKs and a number of adaptor proteins to relay signaling by activating the RAS/MAPK pathway. TNO155 binds the inactive, or “closed” conformation of SHP2, thereby preventing its opening into the active conformation. This prevents the transduction of signaling from activated RTKs to the downstream RAS/MAPK pathway.

TNO155 has demonstrated efficacy in a wide range of RTK-dependent human cancer cell lines and in vivo xenografts. Preclinical in vitro and in vivo evaluation of TNO155 demonstrate selective and potent inhibition of the SHP2 phosphatase, in RTK-dependent human cancer models, for example, EGFR-mutant non-small cell lung cancer (NSCLC); KRAS mutant non-small cell lung cancer; head and neck squamous cell carcinoma (HNSCC); melanoma; gastrointestinal stromal tumors (GIST); colorectal cancer (CRC); medullary thyroid cancers; and ALK-rearranged NSCLC. SHP2 inhibition can be measured by assessing biomarkers within the MAPK signaling pathway, such as decreased levels of phosphorylated ERK1/2 (pERK) and downregulation of dual specificity phosphatase 6 (DUSP6) mRNA transcript. In the KYSE-520 (esophageal squamous cell carcinoma) and DETROIT-562 (pharyngeal squamous cell carcinoma) cancer cell lines, the in vitro pERK IC50's were 8 nM (3.4 ng/mL) and 35 nM (14.8 ng/mL) and the antiproliferation IC50's were 100 nM (42.2 ng/mL) and 470 nM (198.3 ng/mL), respectively. The antiproliferative effect of TNO155 was revealed to be most effective in cancer cell lines that are dependent on RTK signaling. In vivo, SHP2 inhibition by orally-administered TNO155 (20 mg/kg) achieved approximately 95% decrease in DUSP6 mRNA transcript in an EGFR-dependent DETROIT-562 cancer cell line and 47% regression when dosed on a twice-daily schedule. Dose fractionation studies, coupled with modulation of the tumor DUSP6 biomarker show that maximal efficacy is achieved when 50% PD inhibition is attained for at least 80% of the dosing interval.

The epidermal growth factor receptor (EGFR) is an established critical therapeutic target in NSCLCs harboring activating EGFR mutations. Numerous trials with first (e.g. erlotinib, gefitinib) and second (e.g. afatinib, dacomitinib) generation EGFR inhibitors have been conducted in the EGFR-mutant advanced/unresectable NSCLC population, and have consistently demonstrated superior efficacy of EGFR tyrosine kinase inhibitors (TKIs) over chemotherapy in this population. Resistance to 1^{st} generation EGFR TKIs has been shown to arise through the development of an EGFR “gatekeeper” T790M mutation that impairs binding of the TKI, as well as by activation of alternative RTK pathways, including MET and ERBB2 amplification. Clinical trials using 3^{rd} generation, irreversible EGFR inhibitors (e.g., osimertinib, rociletinib), which inhibit EGFR activating and gatekeeper mutations have demonstrated efficacy in EGFR T790M-mutant NSCLCs, highlighting their continued dependence on EGFR signaling. Emerging data from cancers that have become resistant to 3^{rd} generation inhibitors suggest that these cancers continue to select for activated RTK signaling, with resistance mutations in EGFR (C797S) as well as RTK amplifications (MET, ERBB2, FGFR1) having been described. Limited treatment options are available for patients whose cancers have developed resistance to 1^{st}/2^{nd} and 3^{rd} generation EGFR TKIs. Since SHP2 transduces EGFR signaling, and preclinical models have demonstrated a strong correlation between RTK dependence and SHP2 dependence, TNO155 is predicted to provide clinical benefit in these cancers whether resistance is driven by signaling from EGFR or another RTK.

More than 90% of head and neck cancers are characterized by overexpression or amplification of EGFR; amplification/overexpression of other RTKs, particularly FGFRs, and their ligands is also common. Inhibition of EGFR with cetuximab in advanced HNSCCs has also demonstrated clinical benefit, though disease control is not durable. The modest efficacy of EGFR inhibition in HNSCC may be related to compensatory signaling through other RTKs, which would be predicted to be abrogated by SHP2 inhibition with TNO155 treatment. In addition, preclinical testing identified head and neck cancer cells as the lineage with the highest frequency of sensitivity to SHP2 inhibition.

Patients with metastatic or unresectable RTK-driven cancers such as anaplastic lymphoma kinase (ALK)-rearranged NSCLC or stem cell factor receptor (KIT)-mutant gastrointestinal stromal tumor (GIST) derive benefit from molecules directly targeting these RTKs, but resistance to these agents invariably occurs. Mechanisms of resistance frequently include drug-resistant mutations in the targeted RTK and/or activation of bypass RTK pathways; in most cases, further treatment options are limited. Targeting SHP2 with TNO155 is a rational approach in such RTK-dependent cancers.

Nazartinib is a targeted covalent epidermal growth factor receptor (EGFR) inhibitor that selectively inhibits activating (L858R, Exon 19 deletion (ex19del)) mutation(s) of EGFR and the T790M resistance mutations while sparing wild type (WT) EGFR. Nazartinib has been studied in 7 clinical trials. In the first-in-human study of nazartinib in patients with EGFRmutated NSCLC, the recommended Phase II dose of single agent nazartinib was determined to be 150 mg QD (tested in 7 dose levels from 75 mg to 350 mg QD—maximum tolerated dose was not established anti-tumor efficacy was observed at all doses). Promising anti-tumor activity of nazartinib has been demonstrated in both pre-treated and treatment-naïve patients with advanced EGFR-mutated NSCLC.

Patients with EGFR-mutant NSCLC have a high disease control rate with EGFR inhibitors (e.g., erlotinib, gefitinib, osimertinib), but resistance invariably develops. Resistance mechanisms are heterogeneous, but commonly result in restoration of mutant EGFR signaling and/or amplification or overexpression of RTKs other than EGFR (such as MET) or of their ligands. Since SHP2 inhibition impairs signaling via multiple RTKs, the combination of TNO155 with nazartinib has the potential to block multiple heterogeneous resistance mechanisms that may arise in different clones within a tumor, while maintaining inhibition of the initiating driver oncogenic EGFR mutation that exists in every tumor cell. Nazartinib was selected for combination with TNO155 because it is not associated with adverse events of decreased left ventricular ejection fraction. Such events have been described for osimertinib, another 3^{rd} generation EGFR TKI (Tagrisso® prescribing information).

The preclinical data presented in the examples, below, provide in vitro and in vivo evidence that the combination of the SHP2 inhibitor, TNO155 and the EGFR inhibitor, nazartinib, exert a significant combination benefit.

The combination therapy of the present invention is thus expected to bring special benefit, e.g. combining efficacy with tolerability, e.g. with reduced side-effects (e.g., reduced skin toxicity and/or cardiomyopathy), to patients suffering from NSCLC such as: NSCLC (e.g. advanced NSCLC) harboring an activating EGFR mutation and having progressed on standard-of-care (SOC) EGFR tyrosine kinase inhibitor (TKI) (or have no SOC EGFR TKI available) and having progressed on platinum-containing combination chemotherapy; or NSCLC (e.g. advanced NSCLC) harboring a KRAS G12 mutation having progression on SOC; or HNSCC having progressed on platinum-containing combination chemotherapy; or

to patients with the following:

a. NSCLC (e.g. advanced NSCLC) harboring an EGFR TKI-sensitizing EGFR mutation (e.g., exon 19 deletion, L858R), after progression on osimertinib or nazartinib.

b. NSCLC (e.g. advanced NSCLC) harboring an EGFR TKI-sensitizing EGFR mutation (e.g., exon 19 deletion, L858R), after progression on a 1^{st} and/or 2nd generation EGFR TKI (e.g., erlotinib, gefitinib, afatinib) and which has been demonstrated to lack a T790 mutation following progression on these agents.

c. NSCLC (e.g. advanced NSCLC) harboring an EGFR TKI-sensitizing EGFR mutation (e.g., exon 19 deletion, L858R), progressing on osimertinib as the most recent prior therapy (continuing on osimertinib treatment until 2 weeks prior to starting study treatment (and thus osimertinib can be continued during the screening period) or for patients who recently discontinued osimertinib.

### Pharmaceutical Compositions

In another aspect, the present invention provides pharmaceutically acceptable compositions which comprise a therapeutically-effective amount TNO155 and nazartinib, formulated together with one or more pharmaceutically acceptable carriers (additives) and/or diluents. As described in detail below, the pharmaceutical compositions of the present invention may be specially formulated for administration in solid or liquid form, including those adapted for oral administration, for example, drenches (aqueous or non-aqueous solutions or suspensions), tablets, e.g., those targeted for buccal, sublingual, and systemic absorption, boluses, powders, granules, pastes for application to the tongue.

The phrase “therapeutically-effective amount” as used herein means that amount of a compound, material, or composition comprising a compound of the present invention which is effective for producing some desired therapeutic effect in at least a sub-population of cells in an animal at a reasonable benefit/risk ratio applicable to any medical treatment.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations.

As set out above, certain embodiments of the present compounds may contain a basic functional group, such as amino or alkylamino, and are, thus, capable of forming pharmaceutically-acceptable salts with pharmaceutically-acceptable acids. The term “pharmaceutically-acceptable salts” in this respect, refers to the relatively non-toxic, inorganic and organic acid addition salts of compounds of the present invention. These salts can be prepared in situ in the administration vehicle or the dosage form manufacturing process, or by separately reacting a purified compound of the invention in its free base form with a suitable organic or inorganic acid, and isolating the salt thus formed during subsequent purification. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, for example, Berge et al. (1977) “Pharmaceutical Salts”, J. Pharm. Sci. 66:1-19).

The pharmaceutically acceptable salts of the subject compounds include the conventional nontoxic salts or quaternary ammonium salts of the compounds, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like. The pharmaceutically acceptable salt of TNO155, for example, is succinate. The pharmaceutically acceptable salt of nazartinib, for example, is mesylate.

In other cases, the compounds of the present invention may contain one or more acidic functional groups and, thus, are capable of forming pharmaceutically-acceptable salts with pharmaceutically-acceptable bases. The term “pharmaceutically-acceptable salts” in these instances refers to the relatively non-toxic, inorganic and organic base addition salts of compounds of the present invention. These salts can likewise be prepared in situ in the administration vehicle or the dosage form manufacturing process, or by separately reacting the purified compound in its free acid form with a suitable base, such as the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like. (See, for example, Berge et al., supra)

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 0.1 percent to about ninety-nine percent of active ingredient, preferably from about 5 percent to about 70 percent, most preferably from about 10 percent to about 30 percent.

In certain embodiments, a formulation of the present invention comprises an excipient selected from the group consisting of cyclodextrins, celluloses, liposomes, micelle forming agents, e.g., bile acids, and polymeric carriers, e.g., polyesters and polyanhydrides; and a compound of the present invention. In certain embodiments, an aforementioned formulation renders orally bioavailable a compound of the present invention.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution, suspension or solid dispersion in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules, trouches and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants; (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof; (10) coloring agents; and (11) controlled release agents such as crospovidone or ethyl cellulose. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the subject compounds may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions.

When the compounds of the present invention are administered as pharmaceuticals, to humans and animals, they can be given per se or as a pharmaceutical composition containing, for example, 0.1 to 99% (more preferably, 10 to 30%) of active ingredient in combination with a pharmaceutically acceptable carrier.

The compounds of the present invention, which may be used in a suitable hydrated form, and/or the pharmaceutical compositions of the present invention, are formulated into pharmaceutically-acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of the combination of the invention will be that amount of each compound which is the lowest dose effective to produce a therapeutic effect. Such an effective dose will generally depend upon the factors described above.

In another aspect, the present invention provides pharmaceutically acceptable compositions which comprise a therapeutically-effective amount of one or more of the subject compounds, as described above, formulated together with one or more pharmaceutically acceptable carriers (additives) and/or diluents.

### EXAMPLES

### TNO155 and Nazartinib

(3S,4S)-8-(6-amino-5-((2-amino-3-chloropyridin-4-yl)thio)pyrazin-2-yl)-3-methyl-2-oxa-8-azaspiro[4.5]decan-4-amine (TNO155) is synthesized according to example 69 of WO2015/107495, respectively. (R,E)-N-(7-chloro-1-(1-(4-(dimethylamino)but-2-enoyl)azepan-3-yl)-1H-benzo[d]imidazol-2-yl)-2-methylisonicotinamide (nazartinib) is synthesized according to Example 5 of WO2013/184757.

The utility of TNO155 and nazartinib described herein can be evidenced by testing in the following examples.

### Example 1

### Combination Synergy with TNO155 and Nazartinib in EGFR Mutant NSCLC Cell Lines

Human cancer cell lines originated from the CCLE (Cancer Cell Line Encyclopedia) authenticated by single-nucleotide polymorphism analysis and tested for mycoplasma infection using a PCR-based detection technology (IDEXX BioAnalytics) when CCLE was established in 2012. All cell lines used were directly thawed from the CCLE collection stock. All cell lines were cultured in RPMI Medium (ThermoFisher Scientific) except HT-29 (McCoy's 5A), RKO (MEMα), MDST8 (DMEM), A-427 (MEMα) and MIA PaCA-2 (DMEM), supplemented with 10% FBS (VWR). Cell lines were used within 15 passages of thawing and continuously cultured for less than 6 months.

For combination dose matrix assay, 2000 to 3000 cells were seeded in 96-well plates in 80 μL media per well. On the second day, serial dilutions of the two combination agents were subsequently added, each in 20 μL media at 6× of final indicated concentrations. 3 days later, cell viability was measured by the CellTiter-Glo Assay (Promega #G7573). The mean and standard deviation (n=3) of percentages of inhibition (relative to the DMSO-treated control) of compound-treated cells and synergy scores between the two compounds were determined. Synergy scores greater than 2 are considered to have synergistic rather than additive effects.

For immunoblotting: Cells (200,000 to 750,000) in 2 mL growth media were seeded in 6-well plates (Corning, #3506). After 24 hours, cells were treated with compounds or growth factors at the indicated concentrations and duration. Cells were lysed on ice in RIPA buffer (Boston Bioproduct #BP-115) supplemented with 1 mM EDTA and Halt Protease and Phosphatase Inhibitor Cocktail (Thermo Fisher Scientific #1861281). Lysates were centrifuged at 14,000 rpm at 4° C. for 15 minutes and protein concentrations were determined using BCA protein assay (Thermo Fisher Scientific). Equal amount of protein was separated by electrophoresis in NuPAGE 4%-12% Bis-Tris gel (Thermo Fisher Scientific #WG1402BX10), and transferred to nitrocellulose membranes (Bio-Rad, #1704159) for immunoblot with indicated primary antibodies. The bound primary antibodies were visualized using Goat anti-rabbit IgG secondary antibody conjugated with Alexa Fluor 700 and goat anti-mouse IgG secondary antibody conjugated with IRDye 800 CW and scanning with an Odyssey Infrared Imager System (Li-Cor). The following primary antibodies were used: phospho-ERK (Cell signaling Technology #4370), phospho-AKT (Cell signaling Technology #4060), Tubulin (Cell signaling Technology #3873), KRAS (Proteintech #12063-1-AP), phospho-MEK (Cell signaling Technology #9154), phospho-RSK3 (Cell signaling Technology #9348), NRAS (Proteintech #10724-1-AP), HRAS (Proteintech #18925-1-AP), phospho-RB (Cell signaling Technology #8516), Cyclin D1 (Cell signaling Technology #2978), phospho-SHP2 (Abcam #ab62322), Actin (Cell signaling Technology #3700), phospho-CRAF (Cell signaling Technology #9427) and phospho-CSF1R (Cell signaling Technology #3155).

Statistical analyses were performed and curve fit and IC₅₀ values were generated using GraphPad Prism 8 software. Statistical significance was determined using unpaired, paired student t test, or Mann-Whitney test. Significance was set at p=0.05.

PC14 and NCI-H1975 cells were treated with an 8×8 combination matrix of 3-fold serially-diluted nazartinib from 3 μM and TNO155 from 10 μM. After 3 days (PC14) or 6 days (NCI-H1975), cell proliferation was measured using the Cell Titer-Glo® assay and luminescent signals of each dose combination were normalized to that of the DMSO (vehicle control) group. Percentage of growth inhibition is displayed numerically as an 8×8 dose grid. The combination (Loewe excess) synergy score for PC14 cells was 5.12 and the combination synergy score for NCI-H1975 cells was 4.92.

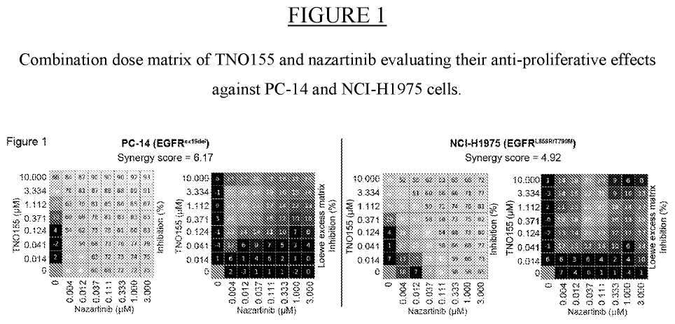

Combination synergy was observed with TNO155 and nazartinib in EGFR mutant NSCLC cell lines, with synergy scores ranging from 2.03 to 5.12 in the cell lines tested (FIG. 1). In PC14 cells, the combination synergy can be attributed to durable pERK inhibition and a higher induction of apoptosis markers such as cleaved poly(ADP-ribose) polymerase (PARP), compared to either single agent nazartinib or TNO155.

Despite the clinical efficacy of EGFR tyrosine kinase inhibitors (TKI) in treating EGFR mutant lung cancers and the significant improvement from the first generation to the third generation EGFR inhibitors, such as sparing WT EGFR, acquired resistance inevitably occurs in the majority of patients. One common mechanism of resistance to EGFR TKI is the acquisition of gatekeeper mutations in EGFR such as T790M against the first generation TKI and C797S against the third generation TKI, which preclude inhibitor binding. Since SHP2 mediates RAS activation downstream of EGFR, the efficacy of SHP2 inhibitors is not affected by the EGFR T790M and C797S mutation, even the two mutations co-occur on the same DNA strand (in cis) as seen in some EGFR T790M patients who relapsed on osimertinib treatment. TNO155 is broadly efficacious in EGFR mutant non-small cell lung cancer (NSCLC) cell lines. Among eight cell lines tested, six were sensitive to nazartinib/EGF816 and TNO155 had activity in three cell lines with IC₅₀ values lower than 1.5 μM (NCI-H3255, HCC827 and PC9 (see table 1):

TABLE 1

| | | | | Nazartinib + |
|---|---|---|---|---|
| | | Nazartinib | TNO155 | TNO155 |
| | EGFR | IC₅₀ | IC₅₀ | Synergy |
| Cell Lines | Mutation | (μM) | (μM) | Score |
| NCI-H3255 | L858R | 0.01 | 0.12 | 3.79 |
| HCC827 | ex19del | 0.04 | 0.70 | 5.94 |
| PC9 | ex19del | 0.01 | 1.46 | 3.39 |
| PC14 | ex19del | 0.05 | 5.01 | 6.17 |
| HCC4006 | ex19del | 0.06 | >10 | 1.59 |
| NCI-H1975 | L858R/T790M | 0.17 | >10 | 4.92 |
| HCC2279 | ex19del | >3 | >10 | 1.15 |
| NCI-H1650 | ex19del | >3 | >10 | 1.52 |

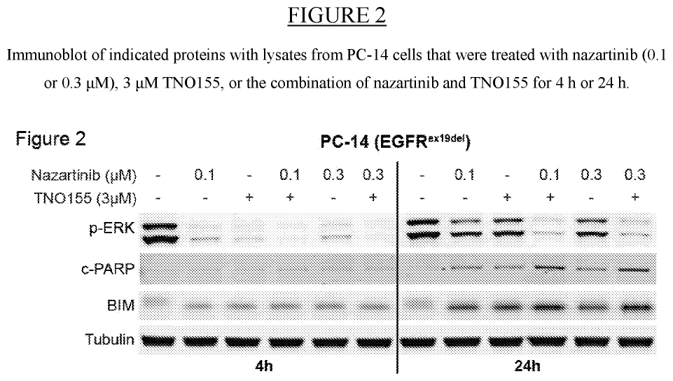

TNO155 synergizes with nazartinib inhibiting EGFR mutant cell proliferation in the combination dose matrix assay. Intriguingly, TNO155 and nazartinib exhibited strong synergy (synergy score >2) in five out of the six nazartinib-sensitive cell lines including two cell lines that were insensitive to TNO155 alone (PC14 and NCI-H1975). The synergy of nazartinib and TNO155 in PC14 and NCI-H1975 cells were observed across a wide range of concentrations of nazartinib and at low concentrations of TNO155 (e.g., 0.124 μM) where TNO155 lacks single agent activity in both lines (see FIG. 1; Loewe excess matrix grids), demonstrating that the contribution of TNO155 comes from the inhibition of alternative RTK signaling that can be feedback activated upon treatment with EGFR TKI. In PC14 cells, a rebound of p-ERK levels was observed after 24 h treatment with 0.1 μM nazartinib following initial suppression, which could not be blocked by a higher dose of nazartinib (0.3 μM) (see FIG. 2). Similarly, TNO155 effectively reduced p-ERK levels at 4 h but also suffered a rebound at 24 h while the combination of TNO155 and nazartinib achieved sustained inhibition of ERK. The combination also induced stronger apoptotic response than either of the single agent at 24 h as evidenced by increased levels of cleaved PARP (c-PARP) and BIM (see FIG. 2).

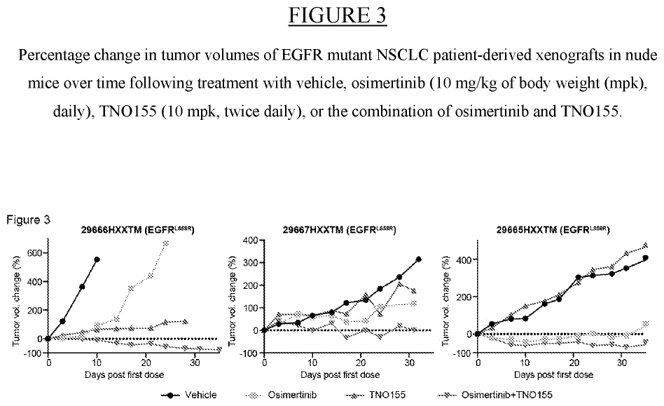

Further, the combination of TNO155 and osimertinib, an FDA-approved third-generation EGFR TKI, was evaluated in a panel of EGFR mutant lung cancer patient-derived tumor models in a mouse clinical trial format and found combination benefits in three EGFR^{(L858R)} models (29666HXXTM, 29667HXXTM and 29665HXXTM). In these models (see FIG. 3), TNO155 was dosed twice a day (BID) due to its short half-life in mice and its maximum tolerated dose is 20 mg per kilogram body weight (mpk). In mouse clinical trial combinations, the dose of TNO155 was reduced to 10 mpk for tolerability reasons with certain combinations. In 29666HXXTM, osimertinib (10 mpk, daily) showed only transient efficacy while TNO155 (10 mpk, BID) effectively slowed down the tumor growth. It was the combination that achieved near complete tumor regression. In 29667HXXTM and 29665HXXTM, osimertinib had modest and strong anti-tumor activity respectively while TNO155 had minimal activity as seen in some EGFR mutant cell lines in vitro, but noticeably enhanced the efficacy of osimertinib. These data suggest that TNO155 can overcome acquired resistance to EGFR TKI and also enhance their efficacy.

### Example 2

Patients were selected with disease amenable to biopsy at baseline, and again during therapy on this study. Patients had either: advanced NSCLC harboring an activating EGFR mutation and having progressed on standard-of-care (SOC) EGFR tyrosine kinase inhibitor (TKI) (or have no SOC EGFR TKI available) and having progressed on platinum-containing combination chemotherapy; or advanced NSCLC harboring a KRAS G12 mutation having progression on SOC; or advanced HNSCC having progressed on platinum-containing combination chemotherapy; or advanced esophageal SCC having progressed on platinum-containing chemotherapy; or advanced CRC lacking activating KRAS (with the exception of KRAS G12C), NRAS, or BRAF mutations and having progressed on fluoropyrimidine, oxaliplatin, and irinotecan; or advanced NRAS/BRAF WT cutaneous melanoma having progressed on SOC; or advanced GIST having progressed on SOC.

Additionally, patients were included with the following:

a. Advanced NSCLC harboring an EGFR TKI-sensitizing EGFR mutation (e.g., exon 19 deletion, L858R), after progression on osimertinib or nazartinib.

b. Advanced NSCLC harboring an EGFR TKI-sensitizing EGFR mutation (e.g., exon 19 deletion, L858R), after progression on a 1^{st} and/or 2nd generation EGFR TKI (e.g., erlotinib, gefitinib, afatinib) and which has been demonstrated to lack a T790 mutation following progression on these agents.

c. Advanced NSCLC harboring an EGFR TKI-sensitizing EGFR mutation (e.g., exon 19 deletion, L858R), progressing on osimertinib as the most recent prior therapy (continuing on osimertinib treatment until 2 weeks prior to starting study treatment (and thus osimertinib can be continued during the screening period). Exceptions can be possible for patients who recently discontinued osimertinib.

The starting dose of nazartinib in this study is 150 mg QD, dosed continuously. In CEGF816X2101, the first-in-human study of nazartinib, doses of nazartinib from 75 mg daily to 350 mg daily were investigated. The maximum tolerated dose was not established, and anti-tumor efficacy was observed at all doses; based on overall safety, tolerability, and efficacy data, 150 mg daily was selected as the recommended dose for the Phase II part of study CEGF816X2101. Thus the selected dose of nazartinib of 150 mg QD is an active dose that is less than half of the highest dose that has been tolerated in patients, thereby allowing an adequate therapeutic window for combination with TNO155. Nazartinib is primarily metabolized by CYP3A4. TNO155 is neither an inducer nor inhibitor of CYP3A4, and thus no effect of TNO155 on nazartinib blood levels is anticipated. The starting dose of TNO155 in combination with nazartinib is 20 mg QD, 2 weeks on/1 week off. Regimens of TNO155 60 mg QD, 2 weeks on/1 week off and 40 mg QD, 3 weeks on/1 week off have been tested and tolerated in patients. Thus the starting dose of 20 mg QD, 2 weeks on/1 week off provides an adequate tolerability margin for combination with nazartinib 150 mg QD.

It is understood that the Examples and embodiments described herein are for illustrative purposes only and that various modifications or changes in light thereof will be suggested to persons skilled in the art and are to be included within the spirit and purview of this application and scope of the appended claims.

What is claimed is:

1. A method of treating cancer comprising administering to a subject in need thereof a pharmaceutical composition comprising (3S,4S)-8-(6-amino-5-((2-amino-3-chloropyridin-4-yl)thio)pyrazin-2-yl)-3-methyl-2-oxa-8-azaspiro[4.5]decan-4-amine, or pharmaceutically acceptable salt thereof, in combination with a second therapeutic agent.

2. The method of claim 1, wherein the cancer is selected from: EGFR-mutant non-small cell lung cancer (NSCLC); KRAS mutant non-small cell lung cancer; head and neck squamous cell carcinoma (HNSCC); melanoma; gastrointestinal stromal tumors (GIST); colorectal cancer (CRC); medullary thyroid cancers; and ALK-rearranged NSCLC.

3. The method according to claim 1 or 2, wherein (3S,4S)-8-(6-amino-5-((2-amino-3-chloropyridin-4-yl)thio)pyrazin-2-yl)-3-methyl-2-oxa-8-azaspiro[4.5]decan-4-amine, or pharmaceutically acceptable salt thereof, and the second therapeutic agent are administered simultaneously, separately or over a period of time.

4. The method according to any one of claims 1 to 3, wherein the amount of (3S,4S)-8-(6-amino-5-((2-amino-3-chloropyridin-4-yl)thio)pyrazin-2-yl)-3-methyl-2-oxa-8-azaspiro[4.5]decan-4-amine, or pharmaceutically acceptable salt thereof, administered to the subject in need thereof is effective to treat the cancer.

5. The method according to any one of claims 1 to 4, wherein the amount of (3S,4S)-8-(6-amino-5-((2-amino-3-chloropyridin-4-yl)thio)pyrazin-2-yl)-3-methyl-2-oxa-8-azaspiro[4.5]decan-4-amine, or pharmaceutically acceptable salt thereof, and the second therapeutic agent, administered to the subject in need thereof, is effective to treat the cancer.

6. The method according to any one of claims 1 to 5, wherein the second therapeutic agent is an EGFR inhibitor.

7. The method of claim 6 wherein the EGFR inhibitor is (R,E)-N-(7-chloro-1-(1-(4-(dimethylamino)but-2-enoyl)azepan-3-yl)-1H-benzo[d]imidazol-2-yl)-2-methylisonicotinamide, or a pharmaceutically acceptable salt thereof.

8. The method according to any one of claims 1 to 7, wherein (3S,4S)-8-(6-amino-5-((2-amino-3-chloropyridin-4-yl)thio)pyrazin-2-yl)-3-methyl-2-oxa-8-azaspiro[4.5]decan-4-amine is administered orally at a dose ranging from about 1.5 to about 100 mg per day, (e.g., from about 1.5 mg to about 60 mg per day, and from about 20 mg to about 60 mg per day).

9. The method according to any one of claims 1 to 8, wherein (3S,4S)-8-(6-amino-5-((2-amino-3-chloropyridin-4-yl)thio)pyrazin-2-yl)-3-methyl-2-oxa-8-azaspiro[4.5]decan-4-amine is administered orally at a dose of about 1.5 mg per day, or 3 mg per day, or 6 mg per day, or 10 mg per day, or 20 mg per day, or 30 mg per day, or 40 mg per day, or 50 mg per day, or 60 mg per day, or 80 mg per day or 100 mg per day.

10. The method of claim 9 wherein (R,E)-N-(7-chloro-1-(1-(4-(dimethylamino)but-2-enoyl)azepan-3-yl)-1H-benzo[d]imidazol-2-yl)-2-methylisonicotinamide is administered orally at a dose ranging from about 75 mg to about 350 mg per day.

11. The method of claim 10, wherein (R,E)-N-(7-chloro-1-(1-(4-(dimethylamino)but-2-enoyl)azepan-3-yl)-1H-benzo[d]imidazol-2-yl)-2-methylisonicotinamide is adminstered orally at about a dose of about 75 mg per day, or 100 mg per day, or 150 mg per day, or 200 mg per day, or 250 mg per day, or 300 mg per day, or 350 mg per day.

12. The method of claim 11, wherein (R,E)-N-(7-chloro-1-(1-(4-(dimethylamino)but-2-enoyl)azepan-3-yl)-1H-benzo[d]imidazol-2-yl)-2-methylisonicotinamide is administered orally at 100 mg or 150 mg per day.

13. The method of claim 11, wherein (R,E)-N-(7-chloro-1-(1-(4-(dimethylamino)but-2-enoyl)azepan-3-yl)-1H-benzo[d]imidazol-2-yl)-2-methylisonicotinamide is administered orally at 150 mg per day.

14. A method of treating cancer comprising administering, to a patient in need thereof, (3S,4S)-8-(6-amino-5-((2-amino-3-chloropyridin-4-yl)thio)pyrazin-2-yl)-3-methyl-2-oxa-8-azaspiro[4.5]decan-4-amine is administered orally at a dose of about 1.5 mg per day, or 3 mg per day, or 6 mg per day, or 10 mg per day, or 20 mg per day, or 30 mg per day, or 40 mg per day, or 50 mg per day, or 60 mg per day, or 80 mg per day or 100 mg per day.

15. The method of claim 14, wherein the dose per day is on a 21 day cycle of 2 weeks on drug followed by 1 week off drug.

16. The method of claim 14 or 15, wherein the cancer is selected from: EGFR-mutant non-small cell lung cancer (NSCLC); KRAS mutant non-small cell lung cancer; head and neck squamous cell carcinoma (HNSCC); melanoma; gastrointestinal stromal tumors (GIST); colorectal cancer (CRC); medullary thyroid cancers; and ALK-rearranged NSCLC.

17. The method of claim 14 further comprising a second therapeutic agent.

18. The method of claim 17 wherein (3S,4S)-8-(6-amino-5-((2-amino-3-chloropyridin-4-yl)thio)pyrazin-2-yl)-3-methyl-2-oxa-8-azaspiro[4.5]decan-4-amine, or pharmaceutically acceptable salt thereof, and the second therapeutic agent are administered simultaneously, separately or over a period of time.

19. The method according to any one of claims 14 to 18, wherein the second therapeutic agent is an EGFR inhibitor.

20. The method of claim 19 wherein the EGFR inhibitor is (R,E)-N-(7-chloro-1-(1-(4-(dimethylamino)but-2-enoyl)azepan-3-yl)-1H-benzo[d]imidazol-2-yl)-2-methylisonicotinamide, or a pharmaceutically acceptable salt thereof.

21. The method of claim 20 wherein (R,E)-N-(7-chloro-1-(1-(4-(dimethylamino)but-2-enoyl)azepan-3-yl)-1H-benzo[d]imidazol-2-yl)-2-methylisonicotinamide is administered orally at a dose of about 75 mg per day, or 100 mg per day, or 150 mg per day, or 200 mg per day, or 250 mg per day, or 300 mg per day, or 350 mg per day.

22. The method of claim 21 wherein (R,E)-N-(7-chloro-1-(1-(4-(dimethylamino)but-2-enoyl)azepan-3-yl)-1H-benzo[d]imidazol-2-yl)-2-methylisonicotinamide is administered orally at 150 mg per day.

23. The method of anyone of claims 1 to 22, wherein the patient or subject is a patient suffering from advanced NSCLC harboring an activating EGFR mutation and having progressed on standard-of-care (SOC) EGFR tyrosine kinase inhibitor (TKI) (or have no SOC EGFR TKI available) and having progressed on platinum-containing combination chemotherapy; or advanced NSCLC harboring a KRAS G12 mutation having progressing on SOC; or advanced HNSCC having progressed on platinum-containing combination chemotherapy; or advanced esophageal SCC having progressed on platinum-containing chemotherapy; or advanced NRAS/BRAF WT cutaneous melanoma having progressed on SOC; or advanced GIST having progressed on SOC.

24. The method of any one of claims 1 to 23, wherein the cancer to be treated is NSCLC which is resistant or refractory to treatment with osimertinib, or a pharmaceutically acceptable salt thereof.

25. A compound for use in a method of treating cancer, wherein the compound is TNO155, or a pharmaceutically acceptable salt thereof and TNO155 is co-administered with nazartinib, or a pharmaceutically acceptable salt thereof.

26. A compound for use in a method of treating cancer, wherein the compound is nazrtinib, or a pharmaceutically acceptable salt thereof and nazartinib is co-administered with TNO155, or a pharmaceutically acceptable salt thereof.

27. The compound for use according to claim 25 or 26, wherein the method is according to any one of claims 1 to 21.

28. A pharmaceutical combination comprising TNO155, or a pharmaceutically acceptable salt thereof, and nazartinib, or a pharmaceutically acceptable salt thereof.
